(19) United States
(12) Patent Application Publication
(10) Pub. No.: US 2015/0209094 A1
(43) Pub. Date: Jul. 30, 2015

(54) POROUS BONE SCREW

(71) Applicant: Biomet Trauma, LLC, Warsaw, IN (US)

(72) Inventor: David Anderson, Winona Lake, IN (US)

(21) Appl. No.: 14/605,012

(22) Filed: Jan. 26, 2015

Related U.S. Application Data
(60) Provisional application No. 61/931,806, filed on Jan. 27, 2014.

(51) Int. Cl. A61B 17/86; B23K 26/34; B23K 15/00

(52) U.S. Cl. CPC A61B 17/864; B23K 15/0086; B23K 26/345; A61B 17/863; A61B 17/866; B33Y 10/00

(57) ABSTRACT

A bone screw includes a core and an outer portion. The core is constructed at least substantially of a porous material. The outer portion is proximate an outer surface of the core and defines a thread portion. The thread portion helically surrounds the core to define a plurality of helical windings such that the core is intermittently exposed axially between adjacent helical windings at least substantially along the length of the thread portion.

### CROSS-REFERENCE TO RELATED APPLICATIONS

This application claims the benefit of U.S. Provisional Application No. 61/931,806, filed on Jan. 27, 2014. The entire disclosure of the above application is incorporated herein by reference.

### FIELD

The present disclosure relates to a bone screw, and more particularly to a bone screw including a porous construct.

### BACKGROUND

This section provides background information related to the present disclosure which is not necessarily prior art.

Various types of surgical procedures may require the use of a bone screw or other fastener to anchor or interconnect a bone or an orthopedic prosthesis to another bone or other tissue. Forces required to maintain a secure connection between the prosthesis and/or the bones may be imparted on, and carried by, the bone screw. While known bone screws have proven to be acceptable for their intended purposes, a continued need for improvement in the art remains. For example, it is desirable to transfer forces, required to maintain a secure connection between the prosthesis and/or the bones, to the bone.

### SUMMARY

This section provides a general summary of the disclosure, and is not a comprehensive disclosure of its full scope or all of its features.

According to one particular aspect, the present disclosure provides a bone screw. A bone screw includes a core and an outer portion. The core is constructed at least substantially of a porous material. The outer portion is proximate an outer surface of the core and defines a thread portion. The thread portion helically surrounds the core to define a plurality of helical windings such that the core is intermittently exposed axially between adjacent helical windings at least substantially along the length of the thread portion.

In some configurations, the outer portion may be monolithically formed from a substantially solid material to include the thread portion, a head, and a tip.

In some configurations, the tip may include an aperture.

In some configurations, the core may include a bore in communication with the aperture.

In some configurations, the head may include a driving feature.

In some configurations, the core may include a bore in communication with the driving feature.

In some configurations, the core may include a proximal end substantially aligned with the head, and a distal end substantially aligned with the tip.

In some configurations, the core may include a bore extending from the proximal end to the distal end.

In some configurations, the tip may include an aperture in communication with the bore.

In some configurations, the outer portion may be formed from a second material that is different than the first material.

In some configurations, the porous material may include a porous metallic material.

In some configurations, the outer portion may include a solid metallic material.

According to another particular aspect, the present disclosure provides a method of manufacturing a bone screw. The method includes forming a substantially porous shaft portion by a first manufacturing process. The method also includes forming a substantially solid outer portion on the shaft portion by a second manufacturing process. The method further includes removing a portion of the solid outer portion to form a head, a helical thread portion, and a tip.

In some configurations, removing a portion of the solid outer portion may include machining a portion of the solid outer portion.

In some configurations, at least one of the first and second manufacturing processes may include an additive manufacturing process.

In some configurations, the additive manufacturing process may include one of an electron beam melting process and a laser sintering process.

In some configurations, the method may include forming a driving feature in the head portion, forming an aperture in the tip, and forming a bore in the shaft portion that is in communication with at least one of the driving feature and the aperture.

According to yet another particular aspect, the present disclosure provides a method of manufacturing a bone screw. The method may include forming a substantially porous shaft portion by a first additive manufacturing process. The method may also include forming a substantially solid outer portion by a second additive manufacturing process. The method may further include coupling the outer portion to the shaft portion.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a bone screw in accordance with the principles of the present disclosure.

FIG. 2 is a perspective view of an outer portion of the bone screw of FIG. 1.

FIG. 3 is a cross-sectional environmental view illustrating the bone screw of FIG. 1 operatively implanted within a bone.

FIG. 4 is a cross-sectional view of a blank of the bone screw of FIG. 1.

FIG. 5 is a cross-sectional environmental view another bone screw operatively implanted within a bone.

Corresponding reference numerals indicate corresponding parts throughout the several views of the drawings.

### DETAILED DESCRIPTION

Example embodiments will now be described more fully with reference to the accompanying drawings.

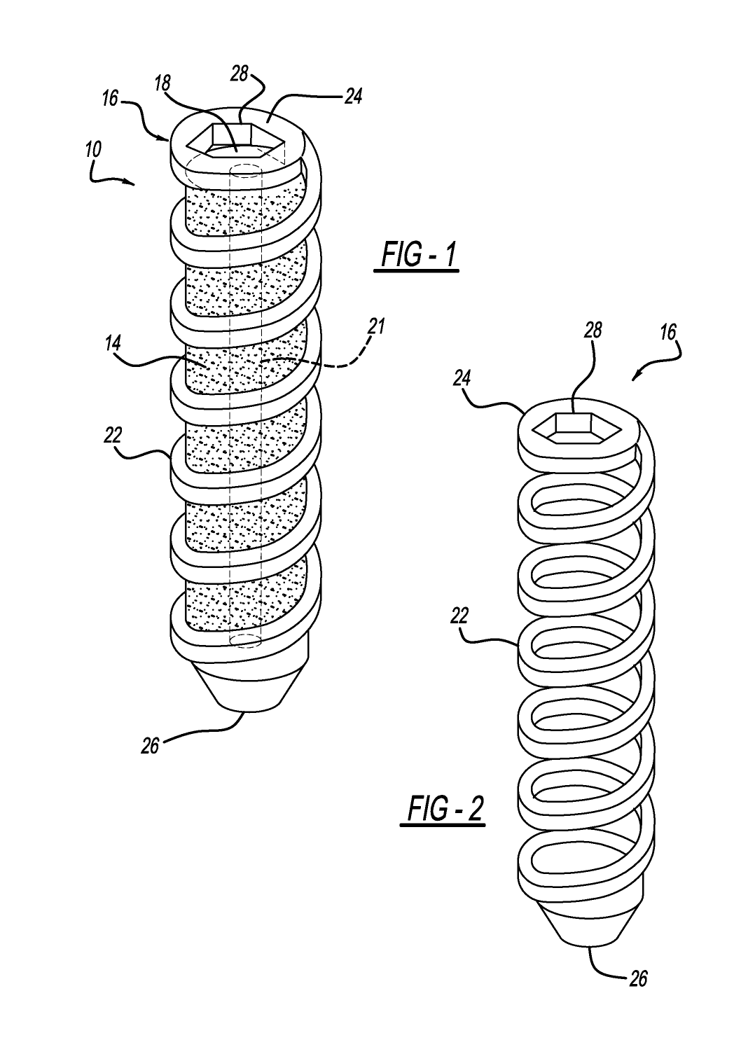

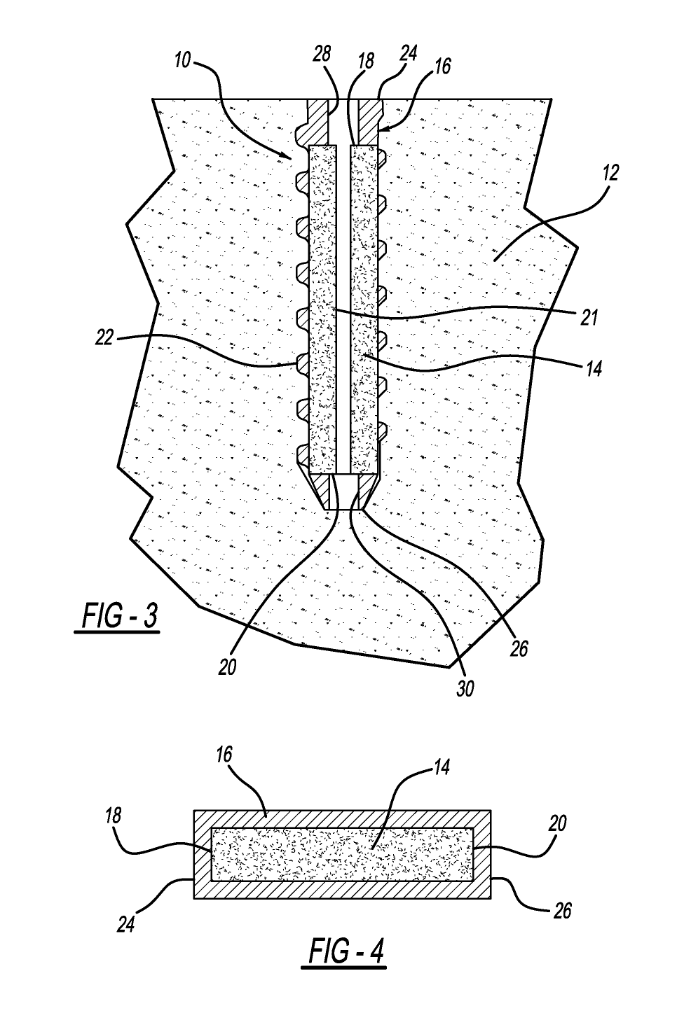

With reference to FIGS. 1-3, a bone screw constructed in accordance with the principles of the present disclosure is illustrated and identified at reference character 10. According to one exemplary use, the bone screw 10 may be used to fix an orthopedic implant (not shown) to a bone 12. It will also be appreciated, however, that the bone screw 10 may be adapted to fix the bone 12 to another bone or tissue.

The bone screw 10 may include a core or shaft portion 14 and an outer portion 16. The shaft portion 14 may be a substantially cylindrical member extending longitudinally from a proximal end 18 to a distal end 20. The shaft portion 14 may be formed from a substantially porous material such as a porous metallic material. In this regard, it will also be appreciated that the shaft portion 14 may also include solid metal portions and/or a bore 21 extending between the proximal and distal ends 18, 20. In one specific configuration, the shaft portion 14 is formed from a porous titanium alloy. It will be appreciated, however, that the shaft portion 14 may be formed from other porous materials such as bone, a ceramic, a polymer, or an epoxy, within the scope of the present disclosure.

The porosity of the shaft portion 14 can allow or improve the ingrowth of the bone 12 into the shaft portion 14 of the bone screw 10. In this regard, it will be appreciated that the shaft portion 14 may provide varying degrees of porosity to promote desired levels of bone ingrowth. As will be explained in more detail below, the ingrowth of the bone 12 into the shaft portion 14 of the bone screw 10 may allow for the distribution or sharing of longitudinal forces between the bone 12 and the outer portion 16 of the screw 10. In this way, the shaft portion 14 can reduce the magnitude of the longitudinal forces imparted on the outer portion 16, which can in turn reduce the potential for future fractures and/or failures of the bone screw 10 and/or of the bone 12 in an area surrounding the bone screw 10.

The outer portion 16 may include a thread portion 22. In the embodiment illustrated, the outer portion further includes a first end or head 24 and a second end or tip 26. The thread portion 22 extends longitudinally from and between the first end or head 24 and the second end or tip 26. The thread portion 22, head 24, and tip 26 may be integrally formed as a monolithic construct. In this regard, the outer portion 16 may be formed from a substantially solid material such as a solid metal material. In one configuration, the outer portion 16 is formed from a solid titanium alloy. It will be appreciated, however, that the outer portion 16 may be formed from other solid materials within the scope of the present disclosure. The solid metal construct of the outer portion 16, alone and/or in combination with the porous metal construct of the shaft portion 14, can provide strength to the bone screw 10 during insertion into, and initial healing of, the bone 12. In this regard, it will also be appreciated that in some configurations, the shaft portion 14 may include a substantially solid construct (e.g., solid metal construct), while the outer portion 16 may include a substantially porous construct (e.g., porous metal construct).

The thread portion 22 may be a helical construct extending around an outer periphery of the shaft portion 14 between the proximal and distal ends 18, 20 thereof. In this regard, the thread portion 22 can define a plurality of helical windings. As such, the core or shaft portion 14 is intermittently exposed axially between adjacent helical windings at least substantially along the length of the thread portion 22. The thread portion 22 may have a uniform or variable pitch and/or major diameter. The head 24 may be located at, and coupled to, the proximal end 18 of the shaft portion 14, and may include driving feature 28. The driving feature 28 may open into and be in communication with the bore 21 of the shaft portion 14. In one configuration, the driving feature 28 may be a hex head. It will be appreciated, however, that the driving feature 28 may include other shapes and configurations (e.g., internal socket) within the scope of the present disclosure. The tip 26 may be located at and coupled to the distal end 20 of the shaft portion 14, and may include an aperture 30 extending therethrough. The aperture 30 may be in communication with the bore 21 of the shaft portion 14. In one configuration, the tip 26 may be conically or frustoconically shaped. It will be appreciated, however, that the tip 26 may have other shapes and configurations within the scope of the present disclosure. For example, the tip 26 may include a drill bit-type shape and configuration.

As will be described in more detail below, the shaft portion 14 and the outer portion 16 may be integrally and coaxially formed or assembled from at least two different materials. In one method of manufacturing the bone screw 10 with a porous metal material, a machineable bone screw blank may be manufactured by utilizing an additive manufacturing process such as laser sintering. The additive manufacturing process may create a porous shaft portion 14 having an outer diameter at least as large as an inner diameter of the outer portion 16 of the bone screw 10. The outer portion 16 may be formed on and around the shaft portion 14 by utilizing an additive manufacturing process such as electron beam melting or laser sintering. In this regard, the porous shaft portion 14 and the solid outer portion 16 can be grown or otherwise built concurrently layer by layer at the same time, such that the bone screw 10 could be grown or constructed layer by layer to form a blank of the bone screw 10 (FIG. 4). Thereafter, the outer portion 16 can be threaded or otherwise machined to create the final shape of the solid external thread portion 22, while exposing the porous shaft portion 14. Alternatively, the bone screw 10 can be grown or otherwise built layer by layer to form the solid external thread portion 22, leaving the porous shaft portion 14 exposed. In yet another alternative manufacturing process, the outer portion 16 may be formed separately as a generally hollow cylindrical construct and thereafter attached to the shaft portion 14. A portion of the outer portion 16 may be machined or otherwise removed to form the thread portion 22, the head 24, and the tip 26. In this manner, a portion of the outer portion 16 may be removed to leave the thread portion 22, the head, and the tip 26, such that the shaft portion 14 is exposed at the inner diameter of the outer portion 16. Machining techniques may include milling, turning, wire electrical discharge machining, etc.

In another method of manufacturing the bone screw 10, the bone screw 10 may be manufactured entirely utilizing an additive manufacturing process. For example, the shaft portion 14 and the outer portion 16, including the thread portion 22, the head 24, and the tip 26, may be laser sintered to a final or near-final shape. Thereafter, the shape and dimensions of the outer portion 16 may be modified utilizing a finishing process such as polishing.

In one method of use, the bone screw 10 is driven into the bone 12 by engaging the head 24 with a driving tool (not shown). Over time, the bone 12 may integrate with the bone screw 10 by growing into the porous shaft portion 14 of the bone screw 10. As forces and loads are imparted in the longitudinal direction through the bone screw 10, a portion of the load is carried by the bone screw 10, including the solid outer portion 16, and another portion of the load is carried by the integrated bone 12 and shaft portion 14 of the bone screw 10. In this way, the ingrowth of bone 12 into the shaft portion 14 can reduce the magnitude of the load carried by the outer portion 16 and thus reduce the probability of fracture or other failure of the bone screw 10 or the bone 12.

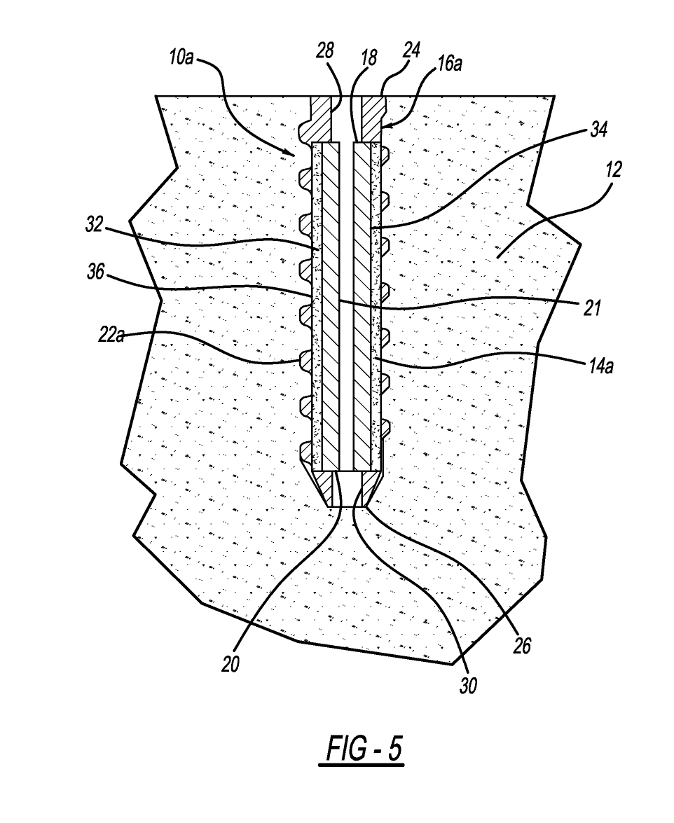

With reference to FIG. 5, another configuration of a bone screw 10a is shown. The structure and function of the bone screw 10a may be substantially similar to that of the bone screw 10 illustrated in FIGS. 1 through 3, apart from any exceptions described below and/or shown in the Figures. Therefore, the structure and/or function of similar features will not be described again in detail. In addition, like reference numerals are used hereinafter and in the drawings to identify like components, while like reference numerals containing letter extensions (i.e., “a”) are used to identify those components that have been modified.

The bone screw 10a may include a core or shaft portion 14a, an intermediate or shell portion 32, and an outer portion 16a. The shaft portion 14a may be a substantially cylindrical member extending longitudinally from the proximal end 18 to the distal end 20. In some configurations, the shaft portion 14a may be formed from a substantially porous material such as a porous metallic material (e.g., porous titanium alloy). It will be appreciated, however, that the shaft portion 14a may be formed from other porous materials such as bone, a ceramic, a polymer, or an epoxy, within the scope of the present disclosure. In other configurations, the shaft portion 14a may be formed from a substantially solid material, such as a solid metallic material (e.g., solid titanium). The shaft portion 14a may also include the bore 21 extending between the proximal and distal ends 18, 20.

The shell portion 32 may be concentrically disposed about an outer peripheral surface 34 of the shaft portion 14a, and the outer portion 16a may be concentrically disposed about an outer peripheral surface 36 of the shell portion 32. In this regard, the shell portion 32 may extend from and between the proximal and distal ends 18, 20 of the shaft portion 14a. In some configurations, the shell portion 32 may be formed from a substantially porous material such as a porous metallic material (e.g., porous titanium alloy). In other configurations, the shell portion 32 may be formed from a substantially solid material, such as a solid metallic material (e.g., solid titanium).

The shaft portion 14a, the outer portion 16a, and the shell portion 32 may be integrally and coaxially formed or assembled from at least two different materials. In some configurations, the shaft portion 14a, the outer portion 16a, and the shell portion 32 may be integrally and coaxially formed or assembled from three different materials. In this regard, in one method of manufacturing the bone screw 10a, an additive manufacturing process may create the shaft portion 14a having an outer diameter at least as large as an inner diameter of the shell portion 32. The shell portion 32 may be formed on and around the shaft portion 14a by utilizing an additive manufacturing process such as electron beam melting or laser sintering. The outer portion 16a may likewise be formed on and around the shell portion 32 by utilizing an additive manufacturing process such as electron beam melting or laser sintering. In this regard, the shaft portion 14a, the outer portion 16a, and the shell portion 32 can be grown or otherwise built concurrently layer by layer at the same time, such that the bone screw 10a could be grown or constructed layer by layer to form a blank of the bone screw 10a (not shown). The outer portion 16a can be threaded or otherwise machined to create the final shape of the external thread portion 22a, while exposing the shell portion 32.

Alternatively, the bone screw 10a can be grown or otherwise built layer by layer to form the external thread portion 22a, leaving the shell portion 32 exposed. In yet another alternative manufacturing process, the outer portion 16a and the shell portion 32 may be formed separately as generally hollow cylindrical constructs. Thereafter, the shell portion 32 can be attached to the outer peripheral surface 34 of the shaft portion 14a, and the outer portion 16a can be attached to the outer peripheral surface 36 of the shell portion 32. A portion of the outer portion 16a may be machined or otherwise removed to form the thread portion 22a, the head 24, and the tip 26. In this manner, a portion of the outer portion 16a may be removed to leave the thread portion 22a, the head 24, and the tip 26, such that the shell portion 32 is exposed at the inner diameter of the outer portion 16a. Machining techniques may include milling, turning, wire electrical discharge machining, etc.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

What is claimed is:

1. A bone screw comprising:
a core constructed of a first material and including a bore; and
an outer portion proximate an outer surface of the core, the outer portion formed from a second material to include a thread portion, a head, and a tip, the head having a first aperture in communication with the bore and the tip having a second aperture in communication with the bore,
wherein the thread portion helically surrounds the core to define a plurality of helical windings such that the core is intermittently exposed axially between adjacent helical windings along the length of the thread portion.

2. The bone screw of claim 1, wherein the outer portion is monolithically formed from the second material.

3. The bone screw of claim 2, wherein the second material includes a solid material.

4. The bone screw of claim 1, wherein the first material includes a porous material.

5. The bone screw of claim 4, wherein the porous material includes a porous metallic material.

6. The bone screw of claim 5, wherein the second material includes a solid metallic material.

7. The bone screw of claim 1, wherein the first material includes a solid material.

8. The bone screw of claim 1, wherein the bore includes a driving feature.

9. The bone screw of claim 1, wherein the core includes a proximal end aligned with the head, and a distal end aligned with the tip.

10. The bone screw of claim 9, wherein the bore extends from the proximal end to the distal end.

11. The bone screw of claim 1, wherein the second material is different than the first material.

12. The bone screw of claim 1, further comprising a shell coaxially disposed between the core and the outer portion.

13. The bone screw of claim 12, wherein the shell is constructed of a third material different than the first and second materials.

14. The bone screw of claim 13, wherein the second and third materials include a substantially porous material, and the first material includes a substantially solid material.

15. A method of manufacturing a bone screw, the method comprising:
forming a porous shaft portion by a first manufacturing process;
forming a solid outer portion on the shaft portion by a second manufacturing process; and
removing a portion of the solid outer portion to form a head, a helical thread portion, and a tip.

16. The method of claim 15, wherein removing a portion of the solid outer portion includes machining a portion of the solid outer portion.

17. The method of claim 15, wherein at least one of the first and second manufacturing processes includes an additive manufacturing process.

18. The method of claim 17, wherein the additive manufacturing process includes one of an electron beam melting process and a laser sintering process.

19. The method of claim 15, further comprising:
forming a driving feature in the head portion;
forming an aperture in the tip; and
forming a bore in the shaft portion that is in communication with at least one of the driving feature and the aperture.

20. A method of manufacturing a bone screw, the method comprising:
forming a porous shaft portion by a first additive manufacturing process;
forming a solid outer portion by a second additive manufacturing process; and
coupling the outer portion to the shaft portion.

21. The method of claim 20, removing a portion of the outer portion to form a head, a helical thread portion, and a tip.
